SEQUENCE LISTING (1 sequence)

SEQ ID NO: 1 - DNA, 6681 bp, Artificial Sequence
  synthetic pNGVL4a-sig/E7(detox)HSP70
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcctcgacggatcttatggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagcttaagtttaaaccgctagcctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

(19) United States
(12) Patent Application Publication
(10) Pub. No.: US 2016/0317641 A1
(43) Pub. Date: Nov. 3, 2016

(54) Methods of Treatment of HPV Related Diseases

(71) Applicant: THE JOHNS HOPKINS UNIVERSITY, Baltimore, MD (US)

(72) Inventors: Tzyy-Choou Wu, Stevenson, MD (US); Chien-Fu Hung, Timonium, MD (US); Richard Roden, Severna Park, MD (US)

(21) Appl. No.: 14/910,350

(22) PCT Filed: Aug. 6, 2014
(86) PCT No.: PCT/US2014/049942

§ 371 Date: Feb. 5, 2016

Related U.S. Application Data
(60) Provisional application No. 61/862,768, filed on Aug. 6, 2013.

(51) Int. Cl. A61K 39/12; C12N 7/00; A61K 31/4745

(52) U.S. Cl. CPC A61K 39/12; A61K 31/4745; C12N 7/00; C12N 2710/20034; A61K 2039/545

(57) ABSTRACT

The present invention provides methods of mucosal tissue administration of therapeutic HPV vaccines, in a prime-boost regimen, which generated antigen-specific CD8+ T cell-mediated immune responses and the expression of tissue-resident memory T cell (Trm) markers on the CD8+ T cells. In some embodiments, the inventive methods employed pNGVL4a-sig/E7(detox)/HSP70 DNA vaccine and TA-HPV in a prime-boost regimen which shows vaccination in the infected mucosal tissues, including those in the cervicovaginal tract, elicited potent antitumor effects and a more effective local immune response in the tissues and regional lymph node, when compared to intramuscular vaccination. Furthermore, targeting the induction of Trm-mediated immune responses can serve as an ideal methodology especially for therapeutic HPV vaccines.

### RELATED APPLICATIONS

This application claims the benefit of priority to U.S. Provisional Patent Application Ser. No. 61/862,768, filed Aug. 6, 2013; the contents of which are hereby incorporated by reference.

### STATEMENT OF GOVERNMENTAL INTEREST

This invention was made with government support under grant nos. CA098252 and CA114425 awarded by NIH. The government has certain rights in the invention.

### BACKGROUND OF THE INVENTION

Human papillomaviruses (HPVs) are the primary etiologic agents of cervical, vulvar, vaginal, penile, oral, throat and anal cancers, and non-oncogenic diseases such as anogenital condyloma or genital warts. Cervical cancer is the third most common female cancer worldwide. The identification of HPV as the etiologic factor for cervical cancer creates an opportunity for developing therapeutic HPV vaccines in order to inhibit the progression of established HPV infection toward HPV precancerous and cancerous lesions. The two HPV viral oncoproteins, E6 and E7, are required for the induction and maintenance of cellular transformation, and are consistently co-expressed in HPV-associated cancers. Thus, they represent ideal targets for the development of a therapeutic HPV vaccine.

Cervical cancer is a cellular alteration that originates in the epithelium of the cervix and is initially apparent through slow and progressively evolving precursor lesions (cervical intraepithelial neoplasia (CIN)), which can be grouped into low and high grade squamous intraepithelial lesions (LSIL and HSIL respectively). 50% of HSIL will eventually progress to cervical cancer. Alterations in cell cycle control mediated by human papilloma virus (HPV) oncoproteins are the main molecular mechanism of action in cervical cancer. HPV infection is very common; the life-time risk for productive women is around 80%. However, most women clear the infection, regardless of HPV type, without experiencing adverse health effects. The most frequently involved HPV types in cervical lesions are HPV 16 and 18, which together cause 70% of cervical cancer cases. Oncogenic HPV infection is a necessary, albeit not sufficient, factor for the oncogenic transformation of cervical-epithelial cells. Additional cofactors, such as an effective immune response leading to viral clearance, determine whether HPV infection will lead to cervical cancer.

As such, there still exists a need for better treatment regimens for HPV related diseases, including cervical cancer.

### SUMMARY OF THE INVENTION

In accordance with an embodiment, the present invention provides a method for generating an immune response against a human papilloma virus (HPV) associated disease in the mucosal tissues of a subject comprising: a) administering to the muscular or mucosal tissues of the subject an effective amount of a composition comprising a first vaccine construct consisting of pNGVL4a-sig/E7(detox)/HSP70; and b) subsequently administering to the mucosal tissues of the subject an effective amount of a composition comprising a second vaccine construct, thereby eliciting an immune response against the HPV infection in the subject.

In accordance with another embodiment, the present invention provides a method for treating cervical cancer in a subject comprising: a) administering to the muscular or vaginal mucosal tissues of the subject an effective amount of a composition comprising a first vaccine construct consisting of pNGVL4a-sig/E7(detox)/HSP70; and b) subsequently administering to the mucosal tissues of the subject an effective amount of a composition comprising a second vaccine construct, thereby eliciting an immune response against the cervical cancer in the subject.

### BRIEF DESCRIPTION OF THE DRAWINGS

FIG. 1 illustrates the characterization of the E7-specific CD8+ T cell immune response using intracellular IFN-g cytokine staining followed by flow cytometry analysis. C57BL/6 mice (5 per group) were vaccinated intramuscularly with pNGVL4a-sig/E7(detox)/HSP70 DNA (50 μg per mouse) prime followed six days later by intraperitoneal injection of TA-HPV (1×10⁷ pfu per mouse) boost, DNA prime followed by DNA boost, TA-HPV only or received no vaccination. One week after the last immunization, splenocytes were analyzed by flow cytometry. Data shown are from a representative flow cytometry analysis. The number in the right upper corner indicates the number of CD8+ IFN-y+E7-specific T cells in 10⁵ total splenocytes.

FIG. 2 depicts the characterization of the E7-specific CD8+ T cells in spleen using E7 peptide-loaded H-2D^{b} tetramer staining. C57BL/6 mice (5 per group) were vaccinated with pNGVL4a-sig/E7(detox)/HSP70 (50 μg per mouse) prime followed six days later by TA-HPV (1×10⁷ per mouse) boost administered either intracervicovaginally (ICV) or intramuscularly (IM). One week after the second immunization, splenocytes were analyzed by flow cytometry. A. Representative flow cytometry analysis. B. Bar graph. Values are shown as mean ±SD, *p<:0.05, **p<:0.01, ns, not significant.

FIG. 3 is the characterization of the E7-specific CD8+ T cells in peripheral blood using E7 peptide-loaded H-2D^{b} tetramer staining. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, blood was analyzed by flow cytometry. A. Representative flow cytometry analysis. B. Bar graph. Values are shown as mean ±SD, *p<:0.05, **p<:0.01, ns, not significant.

FIG. 4 shows characterization of the E7-specific CD8+ T cells in cervicovaginal tract using E7 peptide-loaded H-2D^{b} tetramer staining. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, cervicovaginal tissues were analyzed by flow cytometry. A. Bar graph of the number of CD8+ T cells in the cervicovaginal tract. B. Bar graph of the number of E7-specific CD8+ T cells in the cervicovaginal tract. Values are shown as mean ±SD, *p<:0.05, **p<:0.01, ns, not significant.

FIG. 5 depicts the characterization of the E7-specific CD8+ T cells in the iliac lymph node (ILN) using E7 peptide-loaded H-2D^{b} tetramer staining C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, ILNs were analyzed by flow cytometry. Bar graph showing the number of E7-specific CD8+ T cells in the ILN. Values are shown as mean ±SD, *p<:0.05, **p<:0.01, ns, not significant.

FIG. 6 shows α4β7 and CCR9 expression by E7-specific CD8+ T cells in cervicovaginal tissue. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, cervicovaginal tissues were analyzed by flow cytometry using E7 peptide-loaded tetramer staining A. Bar graph showing α4β7 expression. α4β7 is a surface marker of T cells that binds with MAdCAM-1 in cervicovaginal tissue. B. Bar graph showing CCR9 expression. CCR9 is a chemokine receptor that binds CCL25. Values are shown as mean ±SD, *p<:0.05, **p<:0.01, ns, not significant.

FIG. 7 is the characterization of α4β7 and CCR9 expression by E7-specific CD8+ T cells in the iliac lymph node. C57BL/6 mice (5 per group) were vaccinated with pNGVL4a-sig/E7(detox)/HSP70 (50 μg per mouse) prime followed six days later by TA-HPV (1×10⁷ per mouse) boost either ICV or IM. One week after the second immunization, ILNs were analyzed by flow cytometry using E7 peptide-loaded tetramer staining A. Bar graph showing α4β7expression. B. Bar graph showing CCR9 expression. Values are shown as mean ±SD, *p<:0.05, **p<:0.01, ns, not significant.

FIG. 8 depicts α4β7, CCR9 and CD103 expression by E7-specific CD8+ T cells in cervicovaginal tissue and spleen. C57BL/6 mice (5 per group) were vaccinated with pNGVL4a-sig/E7(detox)/HSP70 (50 μg per mouse) prime followed six days later by TA-HPV (1×10⁷ per mouse) boost by ICV administration. One week after the second immunization, tissue-infiltrating lymphocytes from cervicovaginal tissues and spleens were isolated and analyzed by flow cytometry using CD8, E7 peptide-loaded tetramer and α4β7, CCR9 and CD103 staining The E7 peptide-loaded tetramer positive CD8+ T cells were gated for further analysis of α4β7, CCR9 and CD103 expression. A. Data shown are representative flow cytometry analysis. B. Bar graph showing α4β7, CCR9 and CD103 expression. Values are shown as mean +SD, *p<:0.05, **p<:0.01, ns, not significant.

FIG. 9 shows in vivo therapeutic antitumor effects generated by pNGVL4a-sig/E7(detox)/HSP70 prime and TA-HPV boost via ICV or IM vaccination. C57BL/6 mice (5 per group) were challenged with luciferase-expressing TC-1 cells (2×10⁴ per mouse) in the submucosa of the vagina. One day later, mice were immunized with pNGVL4a-sig/E7(detox)/HSP70 and 6 days later, mice were immunized with TA-HPV. The signal in the vagina was monitored by luminescence on day 7 and day 14 after injection of TC-1 luciferase expressing cells.

FIG. 10 Local and systemic immune responses produced by different vaccination routes. C57BL/6 mice (5 per group) were vaccinated with pNGVL4a-sig/E7(detox)/HSP70 (50 μg per mouse) twice with 7 day interval intramuscularly (IM) or intracervicovaginally (ICV), followed by TA-HPV boost intramuscularly (IM) or intracervicovaginally (ICV) 7 days after the second DNA vaccination. 7 days after the last immunization, mice were sacrificed and splenocytes and cervicovaginal cells were isolated and analyzed by flow cytometry. A. Representative flow cytometry analysis and B, Bar graph showing the number of E7-specific CD8+ T cells in splenocytes. C, Representative flow cytometry and D, Bar graph showing the number of E7-specific CD8+ T cells in the cervicovaginal cells. Values are shown as mean ±SD, *p<0.05, **p<0.01, ns, not significant.

FIG. 11 is a schematic diagram of the pNGVL4a-Sig/E7(detox)/HSP70 plasmid vector used for anti-tumor vaccination.

### DETAILED DESCRIPTION OF THE INVENTION

The inventors have previously employed a DNA vaccine and a vaccinia vaccine targeting E6 and/or E7 for use in HPV-associated disease, including having developed a therapeutic HPV DNA vaccine, pNGVL4a-sig/E7(detox)/HSP70, encoding a chimeric protein consisting of a signal peptide (sig) linked to HPV-16 E7 antigen and heat shock protein 70 (HSP70) (U.S. patent application Ser. No. 10/555,669, and incorporated by reference herein). In addition, pNGVL4a-sig/E7(detox)/HSP70 DNA vaccine has been used in a clinical trials in patients with high grade intraepithelial lesions and proven to be safe (Clin. Cancer Res., 15:361-7 (2009)). Currently, the DNA vaccine, pNGVL4a-sig/E7(detox)/HSP70, is being used in combination with a recombinant therapeutic HPV vaccine, TA-HPV, in the context of an intramuscular DNA prime and vaccinia boost regimen in patients with grade 3 cervical intraepithelial neoplasia (NCT00788164) (Sci. Transl, Med. 6, 221 ra13 (2014).). TA-HPV is a recombinant vaccinia vaccine that encodes HPV-16/18 E6 and E7 proteins. TA-HPV has been used in several clinical trials and proved to be safe (Lancet, 347:1523-7 (1996)). As such, pNGVL4a-sig/E7(detox)/HSP70 DNA vaccine and TA-HPV are favorable for use in a prime-boost regimen.

Recently, tissue-resident memory T cells (Trms) have been shown to play significant roles in local immune responses involved in infection and immunization. As such, site-specific vaccination of the present invention that is able to take advantage of the robust protective immunity provided by Trms is now considered by the present inventors to serve as an ideal methodology especially for mucosal tumors.

The methods of the present invention indicate that it is important to consider a unique strategy to generate Trms through our therapeutic HPV vaccine for the control of HPV-associated diseases occurring in mucosal tissue.

In accordance with some embodiments, the present inventors examined the effects of intravaginal administration of therapeutic HPV vaccines, in a prime-boost regimen, on the generation of antigen-specific CD8+ T cell mediated immune responses systemically and locally and the expression of Trm markers on the CD8+ T cells. pNGVL4a-sig/E7(detox)/HSP70 DNA vaccine and TA-HPV was employed and it was found that intravaginal administration elicited a more effective local immune response in the cervicovaginal tract and regional lymph node. Importantly, the E7-specific CD8+ T cells generated by the prime-boost vaccinations expressed markers of Trms specific for mucosal tissue. Thus, the methods of the present invention show that intracervicovaginal vaccination regimens employing pNGVL4a-sig/E7(detox)/HSP70 DNA and TA-HPV generated potent E7-specific Trm immune responses and antitumor effects against the TC-1 tumor model. As used herein, the term “cervicovaginal” is used interchangeably with the word “vaginal” and includes all muscular and mucosal tissues of the vagina and cervix.

In accordance with an embodiment, the present invention provides a method for generating an immune response against a human papilloma virus (HPV) associated disease in the mucosal tissues of a subject comprising: a) administering to a muscular or mucosal tissue of the subject an effective amount of a composition comprising a first vaccine construct consisting of pNGVL4a-sig/E7(detox)/HSP70; and b) administering to the mucosal tissue an effective amount of a composition comprising a second vaccine construct.

In other embodiments, the present invention provides a method for treating cervical cancer, its precursor lesions and other HPV-associated lesions in a subject in need thereof comprising: a) administering to the muscular or cervicovaginal mucosal tissues of the subject an effective amount of a composition comprising a first vaccine construct consisting of pNGVL4a-sig/E7(detox)/HSP70; and b) administering to said cervicovaginal mucosal tissues an effective amount of a composition comprising a second vaccine construct.

In accordance with some embodiments, the second vaccine construct can be pNGVL4a-sig/E7(detox)/HSP70 or TA-HPV.

In accordance with an embodiment, the second vaccine construct is TA-HPV.

It will be understood by those of ordinary skill in the art that the methods of the invention can be used in many variations of regimens, and should not be limited by any particular example. The vaccination regimen can vary with treatment. In accordance with an embodiment, the second vaccine construct is administered within 5 to 30 days after administering the first vaccine construct. In another embodiment, the second vaccine construct is administered within 6 days after administration of the first vaccine construct.

In some embodiments, the second vaccine construct is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administering the first vaccine construct. In some embodiments, the second vaccine construct is administered less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administering the first vaccine construct.

It will be understood by those of ordinary skill in the art that the present inventive methods are directed to administering the vaccines in the infected mucosal tissues of the subject. In some embodiments, the mucosal tissues are selected from the group consisting of oral mucosa, nasal mucosa, cervicovaginal mucosa and anal mucosa. In an embodiment, the mucosal tissue is the cervicovaginal mucosa.

The human papillomavirus is a DNA tumor virus that causes epithelial proliferation at cutaneous and mucosal surfaces. More than 100 different types of the virus exist, including approximately 30 to 40 strains that infect the human genital tract. Of these, there are oncogenic or high-risk types (16, 18, 31, 33, 35, 39, 45, 51, 52, and 58) that are associated with cervical, vulvar, vaginal, penile, oral, throat and anal cancers, and non-oncogenic or low-risk types (6, 11, 40, 42, 43, 44, and 54) that are associated with anogenital condyloma or genital warts. HPV 16 is the most oncogenic, accounting for almost half of all cervical cancers, and HPV 16 and 18 together account for approximately 70% of cervical cancers. HPV 6 and 11 are the most common strains associated with genital warts and are responsible for approximately 90% of these lesions.

In accordance with an embodiment, the HPV associated disease treated by the inventive methods is cancer, including cervical, vulvar, vaginal, penile, oral, throat and anal cancers. In some embodiments, the cancer is cervical cancer.

In some embodiments, the subject has been diagnosed with a HPV associated disease.

In accordance with some embodiments, the inventive methods comprise vaccination at the cutaneous and mucosal surfaces which are infected with HPV.

In some embodiments, the subject has not been diagnosed with a HPV associated disease.

The vaccination regimen of the present invention can be applied to a subject at least once. In some embodiments the vaccination method is repeated at least once. In some embodiments the vaccination method is not repeated.

In accordance with some embodiments, the inventive methods comprise administering an effective amount of at least one biologically active agent after administering the first vaccine construct. The biologically active agent may be imiquimod (1-(2-methylpropyl)-1H-imidazo[4,5-c]quinolin-4-amine).

In some embodiments, administering a composition comprises injecting the composition. Administering a composition to a mucosal tissue may comprise injecting the composition into the submucosal area of the mucosal tissue.

As used herein, the term “subject” can mean a subject suspected of having cervical cancer or suspected of having an increased risk of having a cervical neoplasia and can include a patient presenting cervical intraepithelial neoplasia (CIN), and/or low grade squamous intraepithelial lesion (LSIL) and/or high grade squamous intraepithelial lesion (HSIL), or any other abnormal Pap smear or cytological test.

As used herein, the term “subject” can also mean a subject suspected of having an HPV infection or HPV related disease, and also includes a subject that has either been exposed to HPV or has evidence of infection with HPV of any variant strain.

As used herein, the term “subject” refers to any mammal, including, but not limited to, mammals of the order Rodentia, such as mice and hamsters, and mammals of the order Logomorpha, such as rabbits. The subject may be from the order Carnivora, including Felines (cats) and Canines (dogs). Alternatively, the subject may be from the order Artiodactyla, including Bovines (cows) and Swine (pigs) or of the order Perssodactyla, including Equines (horses). Alternatively, the subject may be of the order Primates, Ceboids, or Simoids (monkeys) or of the order Anthropoids (humans and apes). The subject may be a human.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

As used herein, the term “treat,” as well as words stemming therefrom, includes diagnostic and preventative treatment, and treatment to improve the subject's condition or at least one symptom of the subject's condition or to prevent the subject's condition or a symptom of the condition from worsening.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a subject or population of subjects. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

In an embodiment, the methods of the present invention can include pNGVL4a-sig/E7(detox)/HSP70 DNA vaccine and TA-HPV in conjunction with a carrier. The carrier is preferably a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. The pharmaceutically acceptable carrier may be one which is chemically inert to the active agent(s) and one which has no detrimental side effects or toxicity under the conditions of use.

The choice of carrier will be determined in part by the chemical properties of pNGVL4a-sig/E7(detox)/HSP70 DNA vaccine and TA-HPV as well as by the particular method used to administer pNGVL4a-sig/E7(detox)/HSP70 DNA vaccine and TA-HPV. Accordingly, there are a variety of suitable formulations of the pharmaceutical composition of the invention. The following formulations for parenteral, subcutaneous, intravenous, intramuscular, intraarterial, intrathecal, intracervicovaginal and intraperitoneal administration are exemplary and are in no way limiting. More than one route can be used to administer pNGVL4a-sig/E7(detox)/HSP70 DNA vaccine and TA-HPV, and in certain instances, a particular route can provide a more immediate and more effective response than another route.

Injectable formulations are in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 14th ed., (2007)).

For purposes of the invention, the amount or dose of pNGVL4a-sig/E7(detox)/HSP70 DNA vaccine and TA-HPV administered should be sufficient to effect, e.g., a therapeutic or prophylactic response, in the subject over a reasonable time frame. The dose will be determined by the efficacy of the pNGVL4a-sig/E7(detox)/HSP70 DNA vaccine and TA-HPV and the condition of a human, as well as the body weight of a human to be treated.

The attending physician may decide the dosage of pNGVL4a-sig/E7(detox)/HSP70 DNA vaccine and TA-HPV with which to treat each individual patient, taking into consideration a variety of factors, such as age, body weight, general health, diet, sex, to be administered, route of administration, and the severity of the condition being treated. By way of example and not intending to limit the invention, the dose of pNGVL4a-sig/E7(detox)/HSP70 DNA vaccine and TA-HPV can be about 1 to 10 mg of pNGVL4a-sig/E7(detox)/HSP70 DNA vaccine and about 1×10⁵ to about 2×10⁷ pfu of TA-HPV to the subject being treated. In some embodiments, the dosage range is about 3 mg of pNGVL4a-sig/E7(detox)/HSP70 DNA vaccine and about 1.6×10⁷ pfu of TA-HPV.

An “active agent” and a “biologically active agent” are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Specific examples of useful biologically active agents the above categories include: anti-neoplastics such as androgen inhibitors, alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; vinca alkaloid natural antineoplastics, such as vinblastine and vincristine.

Other biologically active agents can include peptides, proteins, and other large molecules, such as interleukins 1 through 18, including mutants and analogues; interferons α, γ, and which may be useful for cartilage regeneration, hormone releasing hormone (LHRH) and analogues, gonadotropin releasing hormone transforming growth factor (TGF); fibroblast growth factor (FGF); tumor necrosis factor-α (TNFα); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-α-γ-globulin; superoxide dismutase (SOD); and complement factors, and biologically active analogs, fragments, and derivatives of such factors, for example, growth factors.

In accordance with an embodiment, the biologically active agent is imiquimod (1-(2-methylpropyl)-1H-imidazo[4,5-c]quinolin-4-amine).

In accordance with one or more embodiments, the pNGVL4a-sig/E7(detox)/HSP70 DNA and TA-HPV vaccines are given by injection, i.m., i.p., i.v., subcutaneously, intracervicovaginal, gene gun, etc.

### EXAMPLES

### Materials and Methods for Examples 1-6

Mice. Six- to eight-week-old female C57BL/6 mice were purchased from the National Cancer Institute (Frederick, Md.). All animal procedures were performed according to approved protocols and in accordance with recommendations for the proper use and care of laboratory animals.

Cells. TC-1 cells expressing the HPV16 E6-E7 proteins and the TC-1 cells expressing the firefly luciferase gene (TC-1 luc) were developed in our laboratory and have been described previously (Vaccine, 25:7824-31, (2007)).

Antibodies and tetramer. Fluorochrome-conjugated anti-mouse monoclonal antibodies (Abs) CD8a-APC, CD103-APC, α4β7-APC were purchased from eBiosciences; CD8A-FITC, 7AAD were purchased from BO Pharmingen; CCR9-FITC was purchased from BioLegend; H2D^{b} E-7 tetramer, which allows for the staining of cells that bind the E-7 peptide, was provided by National Institute of Allergy and Infectious Diseases tetramer core facility. Ammonium chloride solution (ACK) was purchased from Quality Biological Inc.

Lymphocyte preparation. Blood was obtained from the tail vessel of the mice and mixed with PBS. Mice were euthanized and organs were removed by dissection. Cervicovaginal (cervical and vaginal tissues) cell suspensions were obtained by enzymatic dispersion in RPMI 1640 digestion buffer for 1 hour at 37° C. while shaking Cervicovaginal cells were passed through a 70-μM cell strainer (Becton Dickinson). Iliac lymph node and spleen cell suspensions were mechanically disrupted and filtered through a 70-μM cell strainer. Blood, cervicovaginal, spleen and lymph node cell suspensions were washed in RPMI/FBS 2% and freed from erythrocytes by treatment with ammonium chloride solution.

Immunization procedures. Mice were immunized by intracervicovaginal or intramuscular routes at day 0 (pNGVL4a-sig/E7(detox)/HSP70 DNA vaccine, 50 μg) and day 7 (TA-HPV 1×10⁷ PFU) with a range of about 1-50 μg/mouse of pNGVL4a-sig/E7(detox)/HSP70 DNA vaccine and 1×10⁶−1×10⁷ PFU/mouse TA-HPV. The total volume injected was 50 μl in both routes. Mice were anesthetized before immunization.

Cell surface staining and flow cytometry analysis. All staining was performed in flow tube in a final volume of 300 μl FACS buffer (PBS+2% FBS) for 1 hour at 4° C. To avoid nonspecific antibody binding through surface Fc receptor, all cells were pre-incubated with CD16/32 mouse BD Fc Block™ (BD pharmingen). Analyses were performed on a Becton-Dickinson FACScan with CELLQuest softare (Becton Dickinson Immunocytometry System, Mountain View, Calif.).

In vivo tumor protection and imaging techniques. About 2×10⁴ TC-1 luc cells were injected into the submucosal area of the genital tract of the mice. Mice were vaccinated on day 2 (pNGVL4a-sig/E7(detox)/HSP70 DNA vaccine) and day 7 (TAHPV) after tumor challenge. Genital tumor growth was monitored by bioluminescence in a Xenogen imaging system once a week. Briefly, D-Luciferin was dissolved to 7.8 mg/mL in PBS, filter sterilized, and stored at −80° C. Mice were given D-Luciferin by i.p. injection (200 μl/mouse, 75 mg/kg) and anesthetized with isoflurane. In vivo bioluminescence imaging for luciferase was conducted on a cryogenically cooled IVIS system using Living Image acquisition and analysis software (Xenogen). Mice were then placed onto the warmed stage inside the light-tight camera box with continuous exposure to 1%-2% isoflurane. Images were acquired 10 minutes after D-luciferin administration and imaged for 2 minutes. The levels of light from the bioluminescent cells were detected by IVIS camera system, integrated, and digitized. Region of interest from displayed images was designated around the cervicovagina tract and quantified as total photon counts using Living Image 2.50 software (Xenogen).

Statistical analyses. All data are expressed as mean ±standard deviation (S.D.) and are representative of at least two separate experiments. Comparisons between individual data points were made using Student's t-test. The non-parametric Mann-Whitney test was used for comparing two different groups. All p values <0.05 were considered significant.

### Example 1

Vaccination with pNGVL4a-sig/E7(detox)/HSP70 DNA vaccine prime followed by TA-HPV boost elicits stronger E7-specific CD8+ T cell response compared to a homologous DNA-DNA prime-boost regimen.

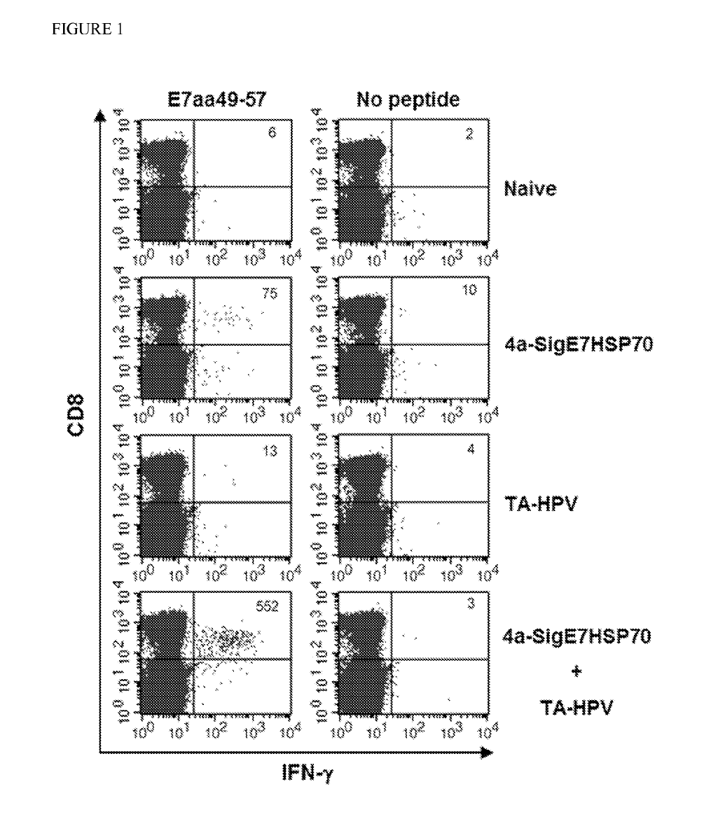

It was first sought to determine the optimal prime-boost vaccination regimen to generate antigen-specific CD8+ T cells using various combinations of the pNGVL4a-sig/E7(detox)/HSP70 DNA and TA-HPV vaccines. C57BL/6 mice (five per group) were vaccinated either with heterologous prime-boost with pNGVL4a-sig/E7(detox)/HSP70 DNA vaccine followed by TA-HPV, homologous prime-boost with pNGVL4a-sig/E7(detox)/HSP70 DNA vaccine, TA-HPV alone or no vaccination. As shown in FIG. 1, the heterologous prime-boost regimen generated the greatest number of IFN-g secreting E7-specfic CD8+ T cells among total splenocytes compared to homologous prime-boost vaccination or TA-HPV alone. This data suggests that DNA priming followed by vaccinia-based boosting is an effective prime-boost regimen to generate activated E7-specific CD8 +T cells.

### Example 2

Vaccination with pNGVL4a-sig/E7(detox)/HSP70 DNA prime followed by TA-HPV boost by intracervicovaginal delivery generates a greater number of E7-specific CD8+ T cells in the cervicovaginal tract compared to vaccination through intramuscular injection.

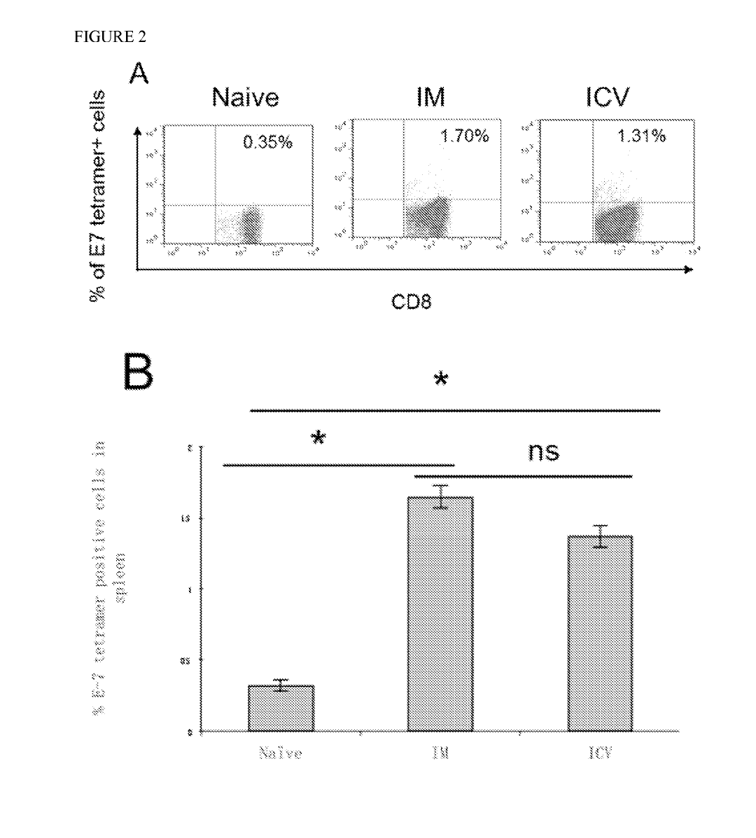

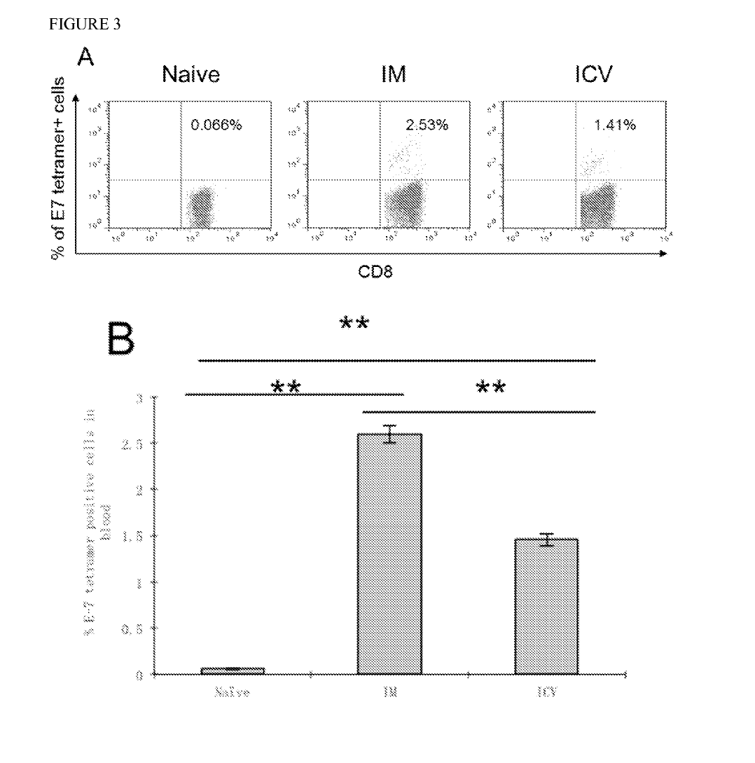

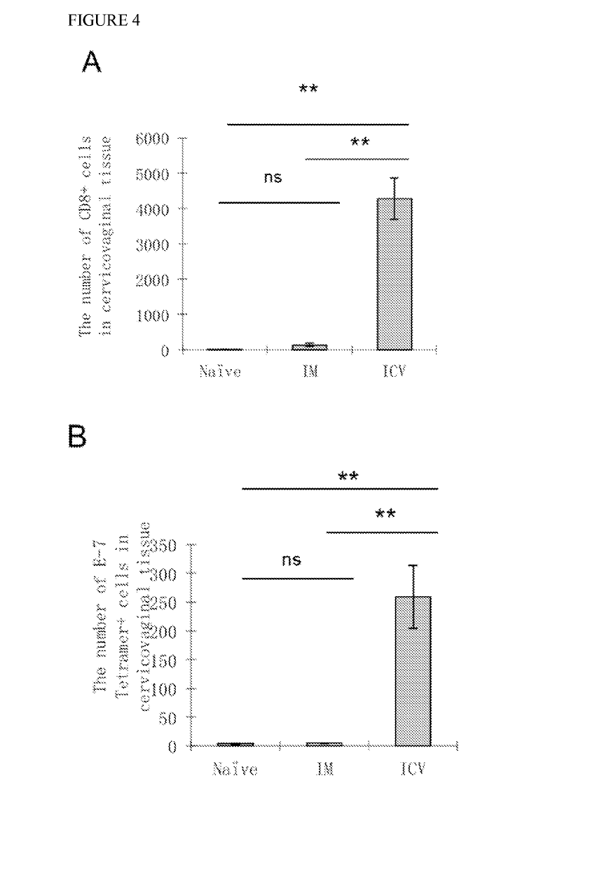

The effect of different administration routes on a vaccination regimen consisting of pNGVL4a-sig/E7(detox)/HSP70 DNA vaccine prime, TA-HPV boost on the generation of antigen-specific CD8+ T cells was examined. C57BL/6 mice were vaccinated either intracervicovaginally (ICV) or intramuscularly (IM) with pNGVL4a-sig/E7(detox)/HSP70 DNA vaccine followed six days later by TA-HPV. One week after TA-HPV vaccination, mice were tested for E7-specific CD8+ T cells in various locations by flow cytometry analysis using E7 peptide-loaded H-2D^{b} tetramer staining As shown in FIG. 2, ICV and IM vaccination with pNGVL4a-sig/E7(detox)/HSP70 DNA vaccine followed by TA-HPV generated significantly higher percentages of E7-specific CD8+ T cells among splenocytes of mice compared to those of naïve mice. However, there appeared to be no significant difference between vaccination through the IM and IVAG routes. FIG. 3 shows that mice vaccinated with IM pNGVL4a-sig/E7(detox)/HSP70 DNA vaccine and TA-HPV generated significantly more E7-specific CD8+ T cells in the peripheral blood compared to mice that were ICV vaccinated. Furthermore, ICV vaccinated mice generated significantly more E7-specific CD8+ t cells than naïve mice. In contrast, ICV vaccination with pNGVL4a-sig/E7(detox)/HSP70 DNA vaccine and TA-HPV induced the highest percentage of E7-specific CD8+ T cells in the murine cervicovaginal tracts compared to IM vaccinated mice and naïve mice (FIG. 4). Taken together, this data indicates that vaccination through intracervicovaginal delivery represents a significantly more efficient method to generate a high number of E7-specific CD8+ T cells in the cervicovaginal tract compared to vaccination through intramuscular injection.

### Example 3

Intracervicovaginal vaccination with pNGVL4a-sig/E7(detox)/HSP70 DNA vaccine prime followed by TA-HPV boost generates a significantly higher number of E7-specific CD8+ T cells in the regional lymph nodes than intramuscular vaccination.

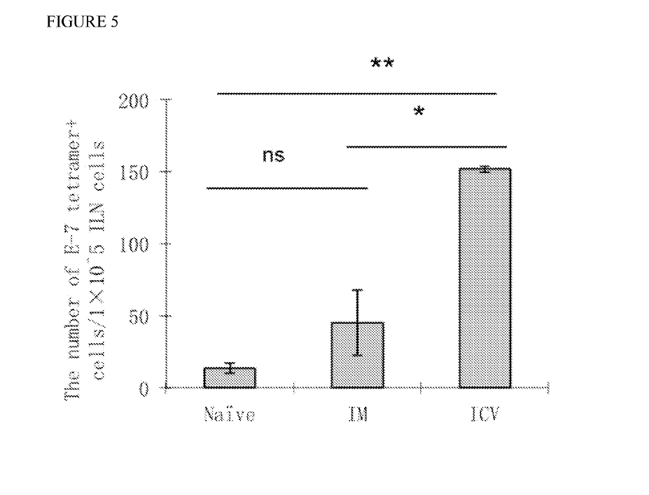

Next, the effect of vaccine administration route on antigen-specific CD8+ T cells in the regional lymph nodes was studied. C57BL/6 mice were vaccinated either ICV or IM with pNGVL4a-sig/E7(detox)/HSP70 DNA vaccine followed six days later by TA-HPV. One week after the last vaccination, the iliac lymph nodes (ILNs) were isolated and tested for E7-specific CD8+ T cells by flow cytometry analysis using E7 peptide-loaded H-2Db tetramer staining. As shown in FIG. 5, the inventors found that mice vaccinated ICV with pNGVL4a-sig/E7(detox)/HSP70 DNA vaccine and TA-HPV had the highest percentage of E7-specific CD8+ T cells in the ILNs compared to mice that were IM vaccinated and naïve mice. These results indicate that ICV vaccination represents a more efficient way to induce a potent local E7-specific cell-mediated immune response compared to IM vaccination.

### Example 4

Intracervicovaginal vaccination with pNGVL4a-sig/E7(detox)/HSP70 DNA vaccine prime followed by TA-HPV boost induces the expression of a4B7 and CCR9 on E7-specific CD8+ T cells.

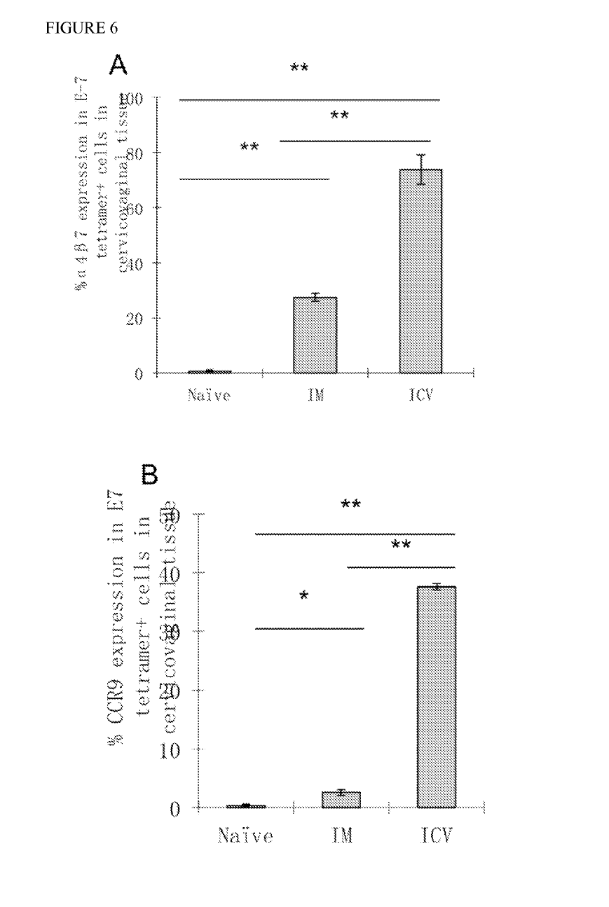

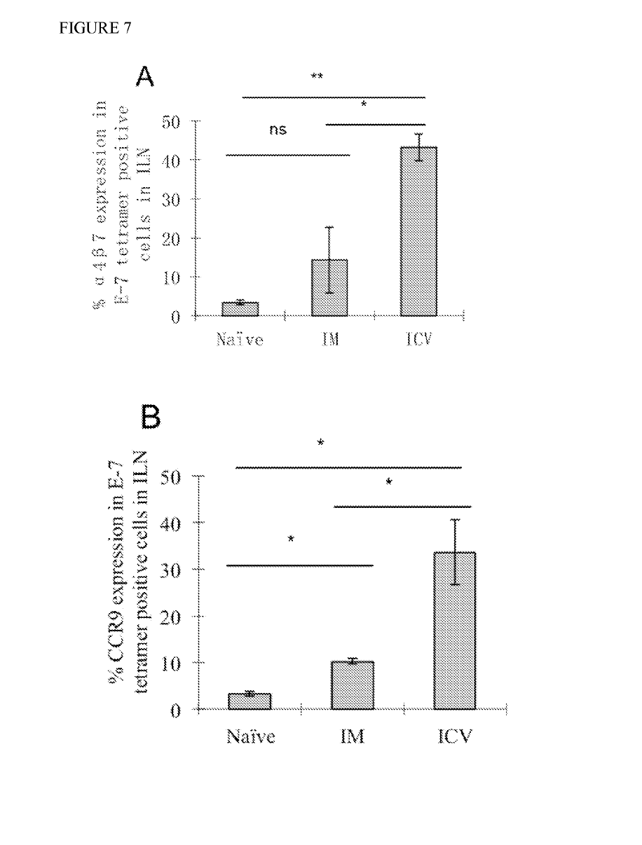

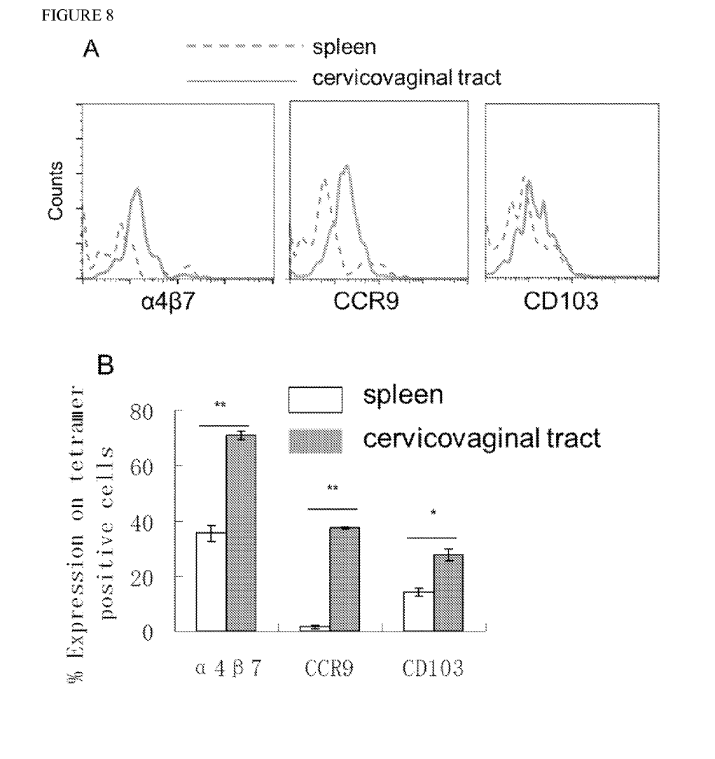

In order to determine whether the E7-specific CD8+ T cells induced by the prime-boost regimen were tissue-resident memory T cells (Trms), the inventors evaluated them for the expression of tissue-specific molecules α4β7and CCR9. α4β7 is a mucosa-associated homing integrin that functions by interacting with Mucosal Addressin Cell Adhesion Molecule-1 (MAdCAM-1). Also involved in the homing and retention of lymphocytes in mucosal tissue is the chemokine receptor CCR9 whose ligand is CCL25, which is commonly expressed in the epithelium of respiratory, gastrointestinal and urogenital tissues. As shown in FIG. 6, mice treated with ICV pNGVL4a-sig/E7(detox)/HSP70 DNA vaccine and TA-HPV had the highest percentage of E7-specific CD8+ T cells expressing a4B7 or CCR9 among all E7 tetramer positive cells in the cervicovaginal tract. Furthermore, ICV vaccinated mice had the highest percentage of E7-specific CD8+ T cells expressing a4β7 or CCR9 in the regional ILN (FIG. 7). These data indicate that ICV vaccination is an effective method to generate antigen-specific CD8+ T cells that express α4β7 or CCR9.

### Example 5

Intravaginal vaccination with pNGVL4a-sig/E7(detox)/HSP70 DNA prime vaccine followed by TA-HPV boost induces the co-expression of α4β7, CCR9 and CD103 on E7-specific CD8+ T cells.

In order to determine the effect of the ICV prime-boost vaccination regimen on mucosal tissue-resident memory T cells (Trms) locally and systemically, the expression of a4β7, CCR9 and CD103 on E7-specific CD8+ T cells in the spleen and cervicovaginal tract of vaccinated mice was examined. Mice were vaccinated ICV with pNGVL4a-sig/E7(detox)/HSP70 DNA vaccine followed by TA-HPV and their splenocytes and cervicovaginal tissues were analyzed by flow cytometry. As shown in FIG. 8, both a4β7 and CCR9 expression on E7-specific CD8+ T cells was significantly higher in the cervicovaginal tract compared to the spleen. This data suggests that ICV vaccination with our DNA-vaccinia prime-boost regimen increases the presence of E7-specific CD8+ T cells that have a surface phenotype consistent with that of mucosal Trms in the cervicovaginal tract.

### Example 6

Intracervicovaginal vaccination with pNGVL4a-sig/E7(detox)/HSP70 DNA prime followed by TAHPV boost generates a significantly improved therapeutic antitumor effect compared to intramuscular vaccination.

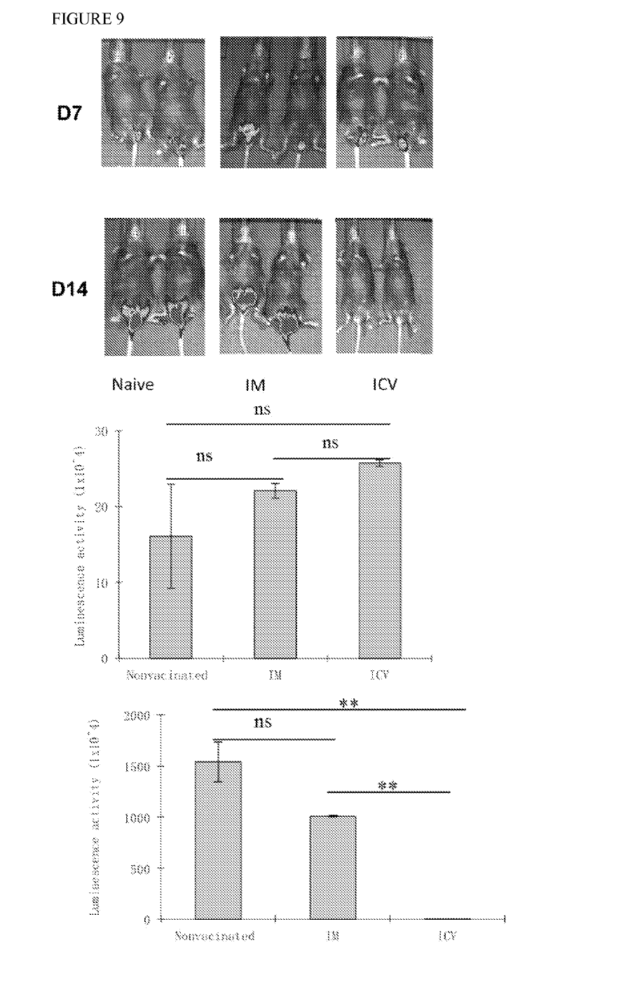

Furthermore, the therapeutic effect of the prime-boost regimen of the present invention administered via different routes was assessed using a luciferase-expressing TC-1 tumor model. The level of luciferase activity represents the tumor load in mice. C57BL/6 mice were challenged subcutaneously with E7—and luciferase-expressing TC-1 tumor cells. One day later, mice were immunized with pNGVL4a-sig/E7(detox)/HSP70 DNA vaccine and 6 days later, mice were immunized with TA-HPV. Mice were monitored for tumor growth by luminescence imaging on day 7 and day 14 after tumor challenge. As shown in FIG. 9, mice receiving ICV vaccination with pNGVL4a-sig/E7(detox)/HSP70 DNA vaccine and TA-HPV experienced significantly greater antitumor effects, as measured by decreased luminescence, on day 14 compared to mice receiving IM vaccination or no vaccination. ICV vaccinated mice had no detectable luminescence on day 14, suggesting that they were eradicated of TC-1 tumor cells. These data indicate that vaccination through ICV is more efficient in generating a potent therapeutic antitumor effect compared to vaccination through IM injection.

### Example 7

Intramuscular pNGVL4a-sig/E7(detox)/HSP70 DNA prime followed by intracervicovaginal TA-HPV boost induces the highest number of E7-specific CD8+ T cells in both the spleen and the cervicovaginal tract.

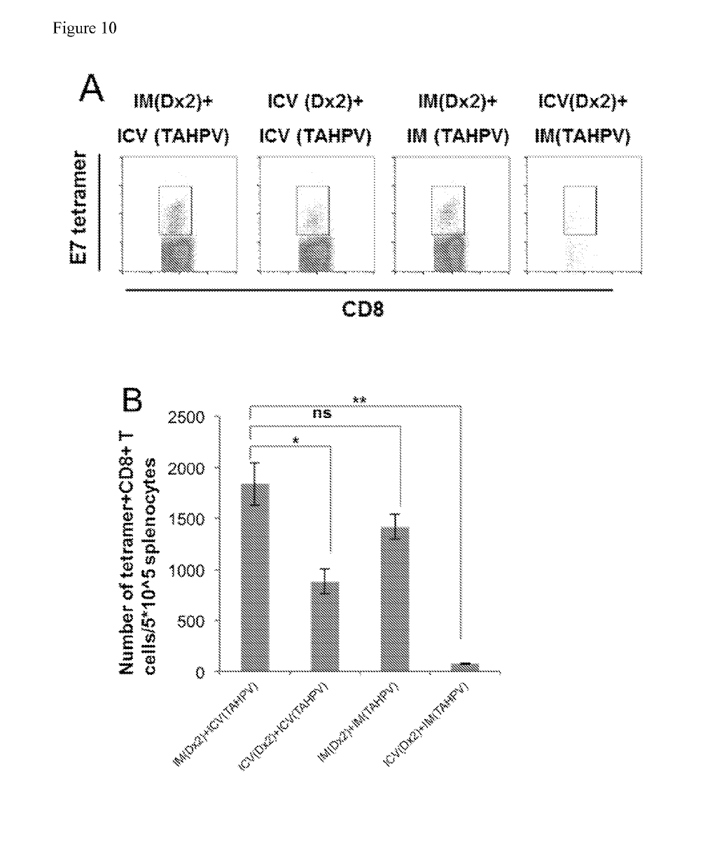

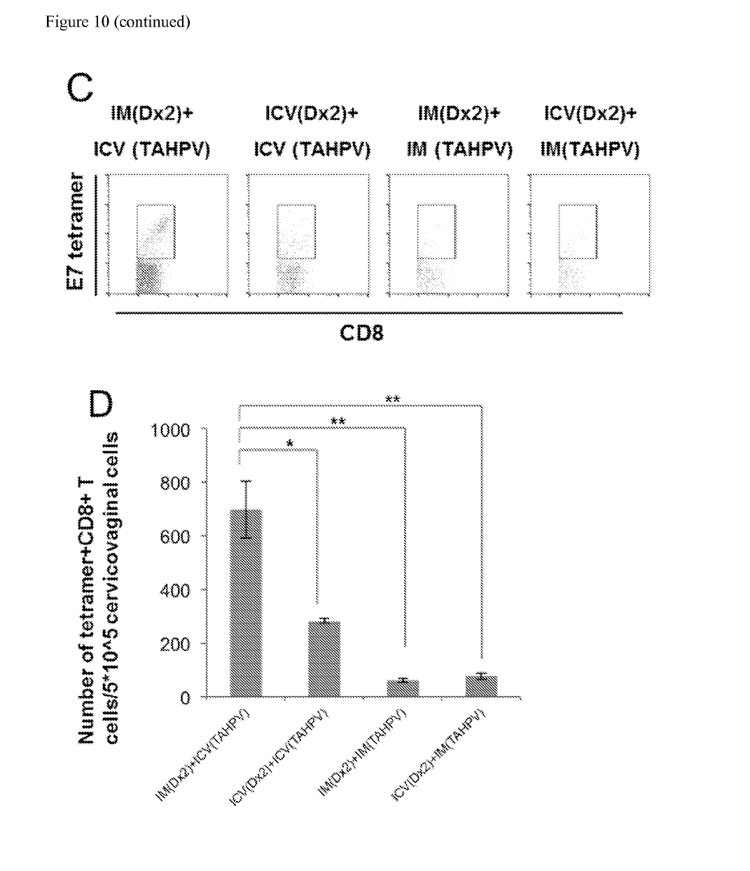

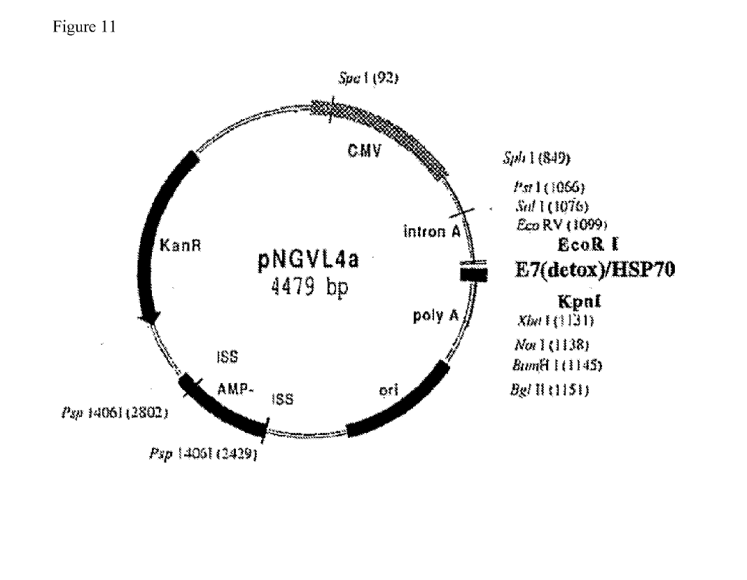

Finally, the systemic (spleen) and local (cervicovaginal tract) HPV E7 specific CD8+ T-cell mediated immune responses induced by different combinations of prime-boost delivery routes were evaluated. C57BL/6 mice (5 per group) were vaccinated with pNGVL4a-sig/E7(detox)/HSP70 DNA (50 μg per mouse) intramuscularly or intracervicovaginally twice with a 7 day interval between vaccinations, followed by intramuscular or intracervicovaginal TA-HPV boost 7 days after the second DNA vaccination. Splenocytes and cervicovaginal cells were harvested and analyzed by flow cytometry 7 days after the last vaccination. FIG. 10 shows that IM DNA priming twice followed by ICV TA-HPV boost triggers the highest E7-specific CD8+ T cell production in both the cervicovaginal tract and in the spleen. Furthermore, ICV TA-HPV boost, regardless of the site of DNA priming, generates superior local production of E7-specific CD8+ T-cells (FIG. 10D). Although IM DNA priming followed by IM TA-HPV boost is effective for the systemic production of CD8+ T cells but this combination is not effective for the generation of local HPV E7-specific CD8+ T cells in the cervicovaginal tract. Taken together, these results suggest that IM DNA prime followed by ICV TA-HPV boost is the most desirable combination to generate HPV E7specific CD8+ T cells both the cervicovaginal tract and in the spleen.

In the current study, the inventors identified that a heterologous DNA-vaccinia prime-boost vaccination regimen is optimal for inducing E7-specific CD8+ T cell immune responses compared to a homologous DNA-DNA prime-boost regimen. The present inventive methods show that the ICV administration route with TA-HPV vaccinia , compared to IM administration, generates greater E7-specific CD8+ T cells in the spleen, peripheral blood, cervicovaginal tract and regional lymph node. Furthermore, the inventors surprisingly found that these E7-specific CD8+ T cells induced by ICV vaccination with therapeutic vaccinia vaccine express the mucosa associated homing integrins a4f37 and CCR9, indicating their consistency with mucosal Trms. Finally, it was demonstrated that mice vaccinated with pNGVL4a-sig/E7(detox)/HSP70 DNA vaccine followed by ICV TA-HPV elicited a significantly more potent therapeutic antitumor effect against TC-1 tumors compared to mice that were vaccinated IM. Taken together, these data indicate that administration of therapeutic HPV vaccines via the intracervicovaginal route of the present invention may be most effective in generating cell-mediated immune responses for the control of HPV-associated disease.

The methods of the present invention show that the administration of therapeutic HPV vaccines via the intrcervicoavaginal route generates antigen-specific CD8+ T cells with the mucosal Trm phenotype as well as potent antitumor effects that are surprisingly superior to those generated by intramuscular vaccination. Furthermore, the present results support the immediate clinical translation of the administration route in a clinical trial employing IM pNGVL4a-sig/E7(detox)/HSP70 DNA prime followed by ICV TA-HPV boost. In conclusion, the results indicate that current therapeutic HPV vaccination regimens can be improved by modifying the vaccination route to induce Trm-mediated immune responses.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Exemplary embodiments of this invention are described herein, including the best mode known to the inventors for carrying out the invention. Variations of those embodiments will be apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the disclosed subject matter. Moreover, any combination of the above-described elements in all possible variations thereof is encompassed by the invention unless otherwise indicated herein or otherwise clearly contradicted by context.

1. A method for generating an immune response against a human papilloma virus (HPV) associated disease in the mucosal tissues of a subject comprising:
a) administering to a muscular or mucosal tissue of the subject an effective amount of a composition comprising a first vaccine construct consisting of pNGVL4a-sig/E7(detox)/HSP70; and
b) administering to said mucosal tissue an effective amount of a composition comprising a second vaccine construct.

2. The method of claim 1, wherein the second vaccine construct is pNGVL4a-sig/E7(detox)/HSP70 or TA-HPV.

3. The method of claim 2, wherein the second vaccine construct is TA-HPV.

4. The method of claim 1, wherein the second vaccine construct is administered within 5 to 30 days after administering the first vaccine construct.

5. The method of claim 4, wherein the second vaccine construct is administered within 6 days after administering the first vaccine construct.

6. The method of claim 1, wherein the tissue is a mucosal tissue selected from the group consisting of oral mucosa, nasal mucosa, cervicovaginal mucosa and anal mucosa.

7. The method of claim 6, wherein the mucosal tissue is the cervicovaginal mucosa.

8. The method of claim 1, wherein the HPV associated disease is cancer or the precursor lesions of cancer.

9. The method of claim 8, wherein the HPV associated disease is cancer, and the cancer is cervical cancer.

10. The method of claim 1, wherein the subject has been diagnosed with a HPV associated disease.

11. The method of claim 10, wherein the subject has been diagnosed with cervical cancer or the precursor lesions of cancer.

12. The method of claim 1, wherein the subject has not been diagnosed with a HPV associated disease.

13. The method of claim 1, wherein administering a composition comprises injecting the composition.

14. The method of claim 1, wherein administering a composition to a mucosal tissue comprises injecting the composition into the submucosal area of the mucosal tissue.

15. A method for treating cervical cancer or the precursor lesions of cancer in a subject in need thereof comprising:
a) administering to the cervicovaginal muscular or mucosal tissues of the subject an effective amount of a composition comprising a first vaccine construct consisting of pNGVL4a-sig/E7(detox)/HSP70; and
b) administering to said cervicovaginal muscular or mucosal tissues an effective amount of a composition comprising a second vaccine construct.

16. The method of claim 15, wherein the second vaccine construct is pNGVL4a-sig/E7(detox)/HSP70 or TA-HPV.

17. The method of claim 16, wherein the second vaccine construct is TA-HPV.

18. The method of claim 15, wherein the second vaccine construct is administered within 5 to 30 days after administering the first vaccine construct.

19. The method of claim 18, wherein the second vaccine construct is administered within 6 days after administering the first vaccine construct.

20. The method of claim 15, wherein the method is repeated at least once.

21. The method of claim 15, wherein the method is not repeated.

22. The method of claim 15, wherein administering a composition comprises injecting the composition.

23. The method of claim 15, wherein administering a composition to a mucosal tissue comprises injecting the composition into the submucosal area of the mucosal tissue.

24. The method of claim 15, comprising administering an effective amount of at least one biologically active agent after administering the first vaccine construct.

25. The method of claim 24, wherein the biologically active agent is imiquimod (1-(2-methylpropyl)-1h-imidazo[4,5-c]quinolin-4-amine).
